(19) United States
(12) Patent Application Publication
(10) Pub. No.: US 2003/0130620 A1
(43) Pub. Date: Jul. 10, 2003

(54) Tube like device that allows deployment via a sidewall defect and method of application

(76) Inventor: Riyadh Alokaili, Charleston, SC (US)

Correspondence Address: RIYADH ALOKAILI, 1742 SAM RITTENBERG BLVD 20G, CHARLESTON, SC, 29407, US

(21) Appl. No.: 10/319,137

(22) Filed: Dec. 16, 2002

Related U.S. Application Data
(60) Provisional application No. 60/346,813, filed on Jan. 10, 2002.

(51) Int. Cl. A61M 5/178

(52) U.S. Cl. 604/160

(57) ABSTRACT

The proposed patents relates to a catheter, tube, dilator, shunt, drain, access device like invention that allows positioning over another catheter, tube, dilator, shunt, wire, cable, drain, access device or the like via a sidewall defect in contrast to the current practice of deployment of catheters, tubes, dilators, drains, access devices or the like over a guiding device such as a wire via the openings at the tips of these devices. This unique method of deployment offers some advantages over current methodology and allows avoiding some limitations with methods in current practice that rely heavily on traditional guiding devices such as wires. Potential advantages include procedural time reduction, less labor-intensive procedures, as well as the ability to perform some objectives not possible with current methods in certain situation.

### BACKGROUND

Endolumenal and minimally invasive medical procedures are increasingly utilized, and as technology advances and better instrumentation and expertise develop the practice will achieve a greater role.

In the medical field procedural time is directly related to radiation of both operator and patient in addition there is an association between the time that a foreign body remains in the patient's body and risks of clotting, stroke, other embolic complications. Observations in the literature have stated that the risk of infection rises with procedural time as well as number of times where the catheter, cable, tube, dilator, shunt, drain, access device, wire or the like are removed and reintroduced in patients due to increased contact with the external environment and increased chance of contamination with micro-organisms. These complications are expected to diminish with reduction of procedural time and degree of manipulation.

In the medical arena a routine part in interventional procedures relies heavily on manipulating wires, catheters, tubes, dilators, shunts, drains, access devices or the like along narrow paths. Frequently during placement of these devices a guiding device such as a wire is positioned into the appropriate site and subsequently a catheter, tube, dilator, drain, access device or the like is advanced over the guiding device to the chosen spot followed by removal of the initially introduced guiding device to allow utilization of the lumen, which initially contained the guiding device.

If there is a need to change the catheter, tube, dilator, drain, access device or the like that is already in place, a guiding device such as a wire is introduced in the catheter, tube, dilator, drain, access device or the like followed by exchange over the guiding device (FIG. 22). These steps occur several times routinely in most procedures adding up to constitute a significant time and effort burden on some if not most procedures.

As explained above the reliance on traditional guiding devices such as wires result in a time and effort penalty it also means that some procedures will be more difficult, less safe or impossible if a traditional guiding device such as a wire can not be introduced for instance because a catheter, tube, dilator, shunt, drain, access device or the like is totally or near totally occluded.

Additionally in the medical field one limitation of the catheters, tubes, dilators, shunts, drains, access devices or the like in current practice is that most catheters, tubes, dilators, shunts, drains, access devices or the like are manufactured to have fixed properties that serve a certain objectives and solve certain obstacles. If a new objective arises or a new obstacle is encountered that the catheter, tube, dilator, shunt, drain, access device or the like is not suited for it is frequent practice in the art to remove it and replace it with a new device that has different properties to solve what the preceding device failed to accomplish. For example a strait catheter may be changed for a curved catheter to deal with an obstacle that the strait catheter was unable to resolve. Ways to modify the catheter in place or the like without removing and replacing it are largely absent.

In summery an alternative to a traditional guiding device such as a wire may offer solutions to current limitations and problems in the art including procedural time shortening, reduction of effort, and ability of performing some procedures that are rendered difficult or impossible by inability to introduce a guiding device such as a wire in the first place. Additionally a mechanism to modify a catheter, tube, dilator, shunt, drain, access device or the like without removal is expected to offer more flexibility, time saving, and potentially cost saving as a new catheter, tube, dilator, shunt, drain, access device or the like with different properties may not be necessary.

### BRIEF SUMMARY OF THE INVENTION

In the medical field it is common practice to use a guiding device such as a wire to exchange catheters, tubes, dilators, drains, access devices or the like (FIG. 22). The proposed inventions will allow exchange of catheters, tubes, dilators, drains, access devices or the like without the need for traditional guiding devices such as wires (FIG. 23). The invention of claim 1 (denoted 4 in figures) is a modification of a catheter, tube, dilator, shunt, drain, access device or the like where a defect along the sidewall is created by design to allow placement, deployment or positioning of the invention of claim 1 uniquely via this sidewall defect in contrast to the current practice of introducing catheters, tubes, dilators, drains, access devices or the like thru openings at the ends.

This will allow application of the device over a catheter, tube, dilator, shunt, drain, access device or the like that is already in place without the need to use a guide wire or the like for exchange. If this is followed by removal of the original catheter, tube, dilator, shunt, drain, access device or the like it effectively has resulted in exchange as the applied device (invention of claim 1 [denoted 4 in figures]) will effectively function as a catheter, tube, dilator, shunt, drain, access device or the like in this case (FIG. 23). Additionally if needed the original catheter, tube, dilator, shunt, drain, access device or the like may be modified while in place by applying the device (invention of claim 1) over the original catheter to modify it as desired without need for a new catheter. For instance this may be used to add rigidity or a curve to a pre-positioned catheter and potentially many other desired modifications.

Although the invention relates directly to the medical field the unique deployment method likely will find application in non-medical fields.

### OBJECTS OF THE INVENTION

It is an object of the present invention of claim 1 (denoted 4 in figures) to provide a tube like device that allows positioning or application of the device into a desired place by being applied to the exterior of a variety of potential guiding devices such as guide wires then advanced over the guiding device.

It is another object of the present invention of claim 1 (denoted 4 in figures) with its new method of application or positioning to allow application or positioning using both traditional guiding devices such as guide wires and devices not intended to be used as guiding devices (such as catheters, tubes, dilators, shunts, drains, access devices or the like) but made suitable for that purpose by the invention of claim 1.

It is another object of the present invention of claim 1 (denoted 4 in figures) after deployment to function as a variety of other devices including devices typically applied over a guiding device such as a wire (i.e. catheters, tubes, dilators, shunts, drains, access devices or the like) with the sole difference in these situation being the optional new method of application or deployment.

It is another object of the present invention of claim 1 (denoted 4 in figures) after deployment to allow a variety of modifications of a device already in place or position without the need for removal of this device. For instance a strait catheter may be modified to a curved catheter by applying the invention of claim 1 or a soft catheter may be made more rigid after applying the invention of claim 1.

It is another object of the present invention of claim 1 (denoted 4 in figures) to become an alternative and function as an access maintenance device or as a guiding device such as guide wires. For instance it will be possible to perform a feeding tube exchange procedure without use of a traditional guiding device such as a wire (FIG. 23).

### BRIEF DESCRIPTION OF THE SEVERAL VIEWS OF THE DRAWINGS

FIG. 1: One potential schematic representation of a cross section through a traditional double lumen catheter, tube, access device or the like. 1—denotes the external surface, 2—denotes the surface of the lumen, 3 and 5 denote the lumens of the double lumen catheter, tube, access device or the like and 8—is the catheter, tube, access device or the like. Attention is drawn to the external (1) and lumenal (2) surfaces of the traditional double lumen catheter, tube, access device or the like and absence of a sidewall defect or break.

FIG. 2: Cross sectional representation of one potential design of invention stated in claim 1. 1—denotes the external surface of the device, 2—denote surface of lumen 5. 3 and 5 denotes two separate lumens, 4—denotes the solid component of the device of claim 1, 6—points to the sidewall defect that allows surfaces 1 and 2 to be continuous on cross sectional images. The sidewall defect (6) may run thru the whole length of the device or part of it. This (as described elsewhere) will allow to accept a catheter, tube, dilator, wire, cable, shunt, drain, access device or the like through the sidewall slit or defect into the lumen as opposed to the traditional method of gaining access to the lumen thru the openings at the ends of catheters, tubes, dilators, shunts, drains, access devices or the like. Lumen 5 is the lumen designed to accept the catheter, tube, dilator, wire, cable, shunt, drain, access device or the like. After being accepted within lumen 5 they will function as a guiding device to deliver the invention of claim 1. Lumen 3 is an additional optional lumen that will have multiple functions including serving as a port for introduction of wires, catheters or other types of minimally invasive devices or materials well known to the art.

FIG. 3: This is one potential cross sectional schematic representation of the device of claim 1 with a traditional catheter, tube, dilator, shunt, drain, access device or the like deployed in lumen 5. 7—denotes the lumen of the traditional catheter, tube, dilator, shunt, drain, access device or the like; 8—denotes the traditional catheter, tube, dilator, shunt, drain, access device or the like.

FIGS. 4-6: Show several potential cross sectional representations of device described in claim 1 where the free edges of the defect (No# 6) are not just opposing surfaces but are designed to overlap or interlock. The overlap may be obtained by different designs some examples are shown in the figures. The creation of the overlap may offer more secure containment of a catheter, tube, wire, cable, dilator, shunt, drain, access device or the like within lumen NO# 5 of the device of claim 1, in addition these designs may offer close approximation of the free edges bordering the defect (number 6) to the degree of being able to contain fluid or gas without leakage to enable infusing or withdrawing fluid or gas through this more secure lumen.

FIG. 7: Showes a longitudinal section of one potential design through devices of claims 1(no# 4) and 2(no# 10), and a traditional catheter, tube, dilator, shunt, drain, access device or the like while fully deployed. 10 denotes invention application device (claim 2), 11 and 12 denotes the lumens of device of claim 2 that accept device of claim 1, number 13 denotes the projecting portion of device 10 which engages within the lumen (No# 5) of the invention in claim 1. 14 denote the lumen of apparatus 10, which will contain both the traditional catheter, tube, dilator, shunt, drain, access device or the like, and the invention of claim 1 (no# 4) after full deployment. 20 denotes the point where the distal end of the invention of claim 1 is full deployed around the traditional catheter, tube, dilator, shunt, drain, access device or the like, 21 denotes the point where the free edges of the sidewall slit or defect are approximated or sealed, and 22 denotes the point where the sidewall defect are separated. The device of claim 2 will have an open and locked state, the device also has locking components. One potential design is denoted 23 and 24.

FIG. 8: Shows a longitudinal section of one potential design through the inventions of claims 1 and 2 and the traditional catheter, tube, dilator, shunt, drain, access device or the like as the edges of the sidewall slit or defect are re-approximated or engaged (number 21)

FIG. 9: Shows a longitudinal section of one potential design through the inventions of claims 1 and 2 and the traditional catheter, tube, dilator, shunt, drain, access device or the like are in place where the distal aspect of the sidewall slit or defect is separated but not yet deployed around the traditional catheter, tube, dilator, shunt, drain, access device or the like.

FIG. 10: Shows a longitudinal section of one potential design through the inventions of claims 1 and 2 and the traditional catheter, tube, dilator, shunt, drain, access device or the like are in place where the distal aspect of the sidewall defect is being forced to separate at point marked by number 22.

FIG. 11: Shows a longitudinal section of one potential design through the inventions of claims 1 and 2 and a traditional catheter, tube, dilator, shunt, drain, access device or the like are in place where the invention of claim 1 has entered the initial receiving port of invention of claim 2 (denoted 10 in figures). At this point the sidewall slit or defect remains closed or approximated.

FIG. 12: Shows a longitudinal section of one potential design through the inventions of claims 1 and 2 and the traditional catheter, tube, dilator, shunt, drain, access device or the like are in place where the invention of claim 1 is barely introduced into its receiving port.

FIG. 13: Cross sectional representation of one potential design of inventions of claim 1 and 2 and the traditional catheter, tube, dilator, shunt, drain, access device or the like in which the elements are fully deployed at plain A where the edges of the sidewall defect have not yet been separated.

FIGS. 14-17: Cross sectional representation of one potential design of inventions of claim 1 and 2 and the traditional catheter, tube, dilator, shunt, drain, access device or the like where the elements are fully deployed at plans B, C, D and E where the edges of the sidewall slit or defect have been separated.

FIG. 18: Cross sectional representation of one potential design of the inventions where the elements are nearly fully deployed at plain F. The free edges bordering the sidewall defect are approximated to each other but not locked at this plane.

FIG. 19: Cross sectional representation of one potential design of inventions of claim 1 and 2 and the traditional catheter, tube, dilator, shunt, drain, access device or the like where the elements are fully deployed at plane G. At this plane the free edges of the sidewall defect are re-approximated and in this diagram the particular design allows the edges to be sealed by the interlocking configuration.

FIG. 20: Demonstration of a sinusoidal design of the sidewall defect or slit (one potential non-linear design of the sidewall defect or slit).

FIG. 21: Demonstration of a spiral design of the sidewall defect (one potential non-linear design of the sidewall defect or slit).

FIG. 22: Demonstrate steps of the traditional method to exchange a catheter, tube, drain, access device or the like with a traditional access maintainance or guiding device such as a guide wire.

FIG. 23: Demonstrate steps of the proposed new method to exchange a catheter, tube, drain, access device or the like. a—denotes the original device destine to be removed. b—denotes the new device (invention of claim 1).

FIGS. 24-27: Showes a longitudinal section of one potential design through devices of claims 5(no# 28) and 2(no#10), and a traditional catheter, tube, dilator, shunt, drain, access device or the like. The illustration demonstrates variable degrees of deploymet along the long axis of the devices. 10 denotes invention application device (claim 2), 21 denotes the point where the free edges of the sidewall slit or defect are approximated or sealed. 3 denotes a secondary optional lumen. The device of claim 2 will have an open and locked state, the device also has locking components(not illustrated).

FIGS. 28 & 29: Showes an external schematic representation of one potential design through devices of claims 5(no# 28) and 2(no#10), and a traditional catheter, tube, dilator, shunt, drain, access device or the like. 11 and 12 denotes the lumens of device of claim 2 that accept device of claim 5. 26 denotes the portion of device of claim 2 that contains the guiding device (catheter, tube, drain, shunt, access device or the like) and the invention of claim 5 after it has accepted the guiding device in its lumen.

FIG. 30: Longitudinal sectional representation of one potential design of inventions of claim 5 and 2 and the traditional catheter, tube, dilator, shunt, drain, access device or the like in which the elements are seen to have variable degrees of deployment at plain A.

FIGS. 31, 32 & 33: Cross sectional representation of one potential design of inventions of claim 5 and 2 and the traditional catheter, tube, dilator, shunt, drain, access device or the like where the elements are deployed at plans B, C and D where the edges of the sidewall slit or defect can be seen in there open and closed states.

Figure Key

1—External surface of device of claim 1

2—Internal surface of the primary lumen of device of claim 1

3—Secondary optional lumen of device of claim 1

4—Invention of claim 1

5—Primary lumen of device of claim 1

6—Points to the defect between surfaces 1 and 2 (sidewall defect)

7—Lumen of the traditional catheter, tube, dilator, shunt, drain, access device or the like

8—Traditional catheter, tube, dilator, shunt, drain, access device or the like

10—Invention application device (device of claim 2)

11&12—Denotes the lumens of device of claim 2 that accept device of claim 1

13—Denotes the projecting portion of device 10 that engages within the lumen (No# 5) of the invention in claim 1.

14—Denote the lumen of apparatus 10 which will contain both the traditional catheter, tube, dilator, shunt, drain, access device or the like and the invention of claim 1 (no# 4) after full deployment.

20—Denotes the point where the distal end of the invention of claim 1 after full deployment around the traditional catheter, tube, dilator, shunt, drain, access device or the like.

21—Denotes the point where the free edges of the sidewall defect are approximated or sealed.

22—Denotes the point where the sidewall defect is separated.

23—Denotes the pivot that allows the components of device in claim 2 (denoted 10) to open and close to receive the traditional catheter, tube, dilator, shunt, drain, access device or the like (denoted 8).

24—Denotes the lock that keeps the device of claim 2 (denoted 10) locked after it has received the traditional catheter, tube, dilator, shunt, drain, access device or the like (denoted 8)

25—Denotes the portion of device of claim 2 that contains the guiding device (catheter, tube, drain, shunt, access device or the like) before being accepted in the lumen of invention of claim 1.

26—Denotes the portion of device of claim 2 that contains the guiding device (catheter, tube, drain, shunt, access device or the like) and the invention of claim 1 after it has accepted the guiding device in its lumen.

27—Denotes the portion of device of claim 2 that receives invention of claim 1, opens its lumen, and closes it over the guiding member(catheter, tube, drain, shunt, access device or the like).

28—Denotes device of claim 5.

### DETAILED DESCRIPTION OF THE INVENTION

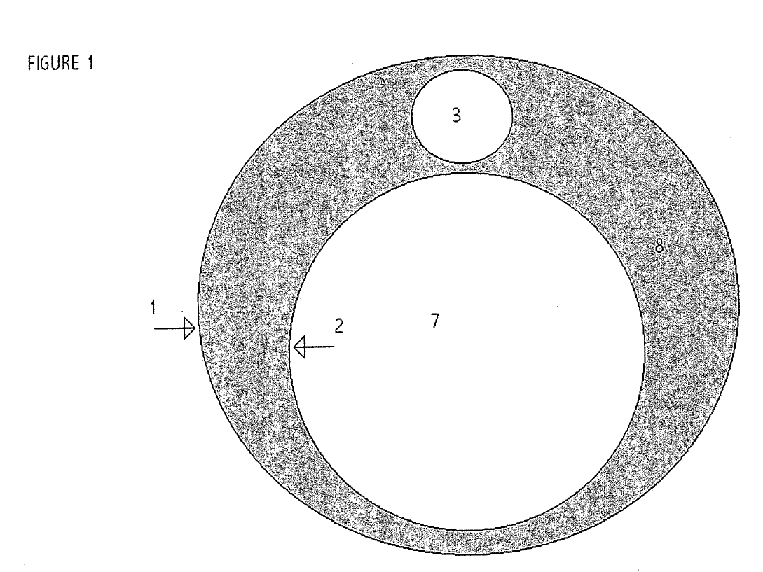

The invention of claim 1 (denoted 4 in figures) is similar to catheters, tubes, dilators, shunts, drains, access devices or the like, devices well known to the art (FIG. 1). They are typically oblonged hollow devices usually cylindrical in morphology with a variety of designs and components that may include several morphological configurations, utilization of multiple options of manufacturing materials, one or more lumens, embedded reinforcement elements, inflatable components, and/or ports for delivery of an ever increasing number of devices and materials all known to the art.

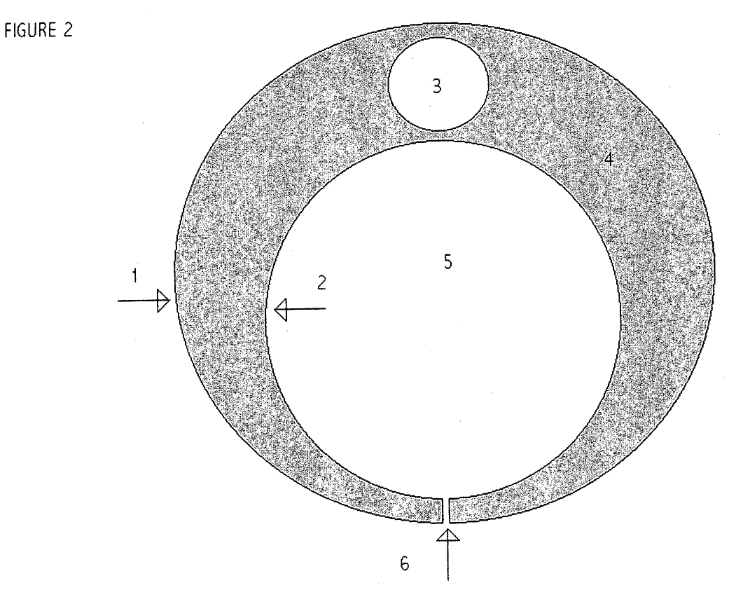

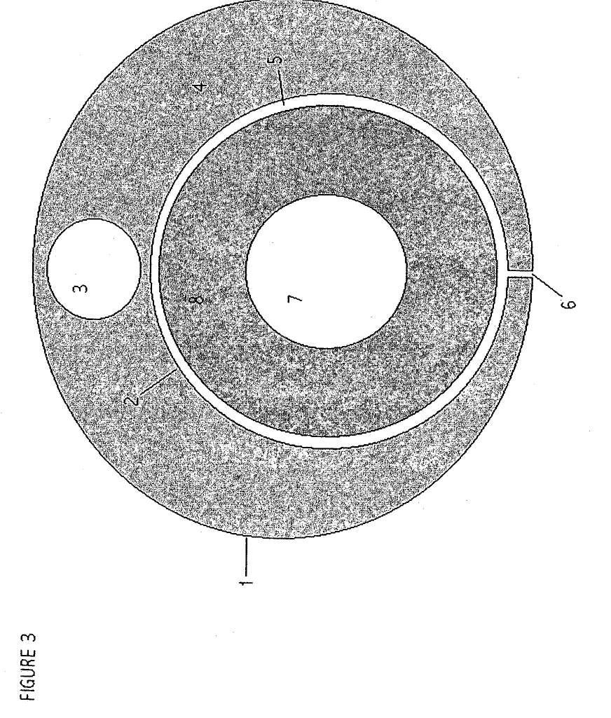

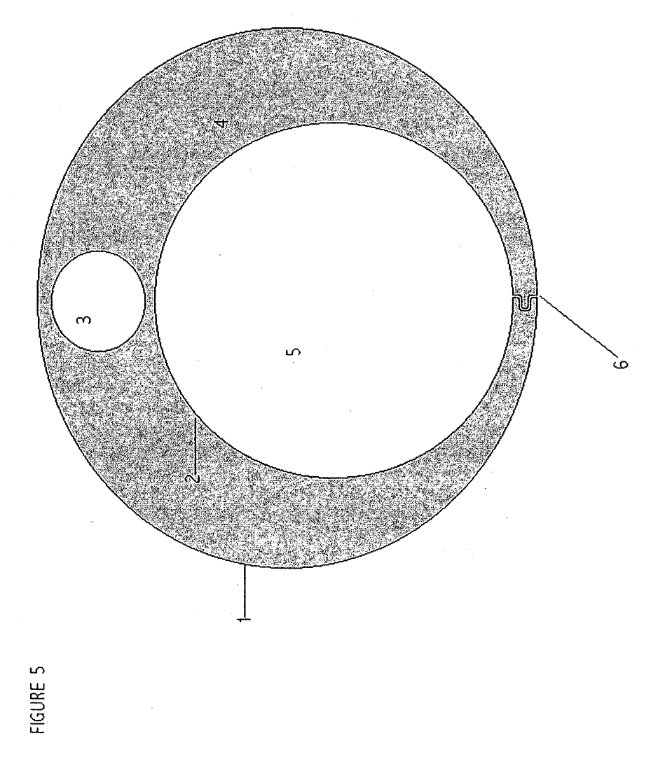

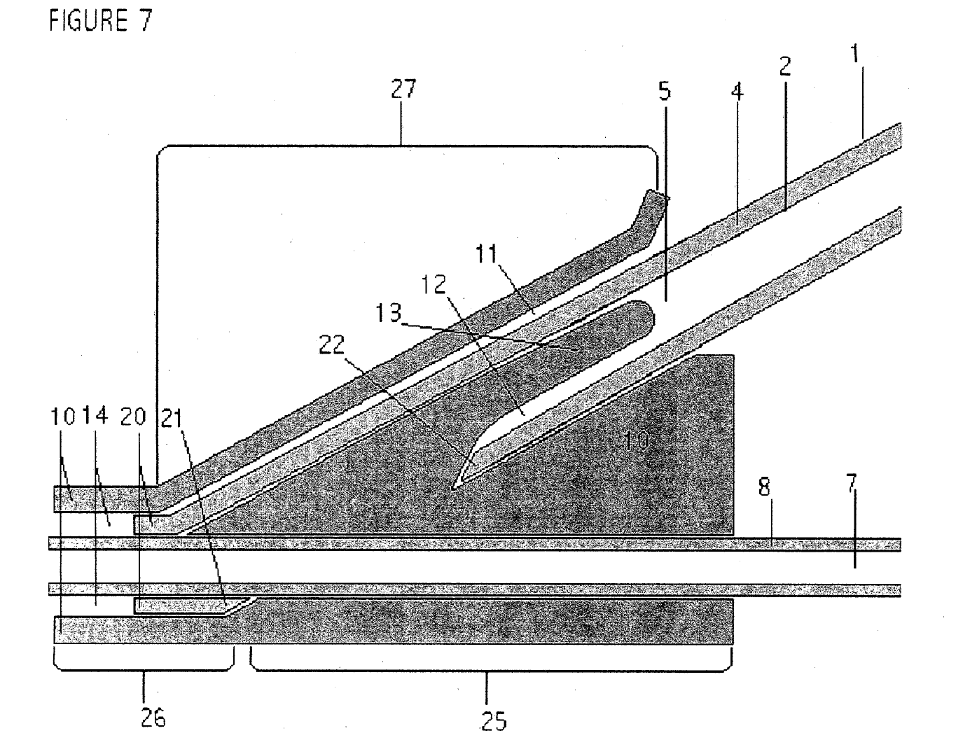

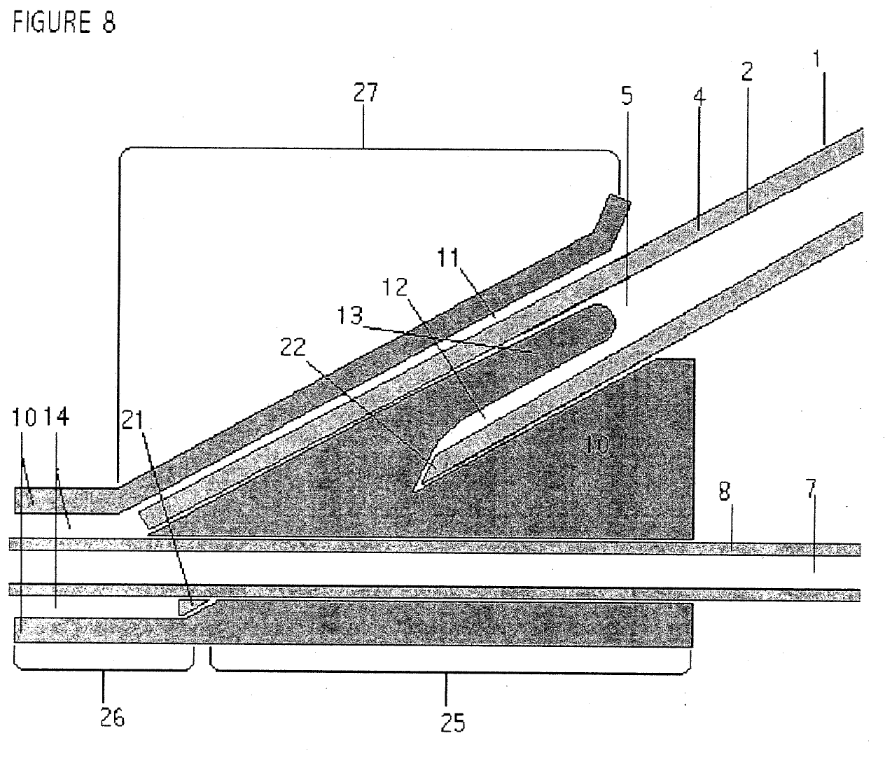

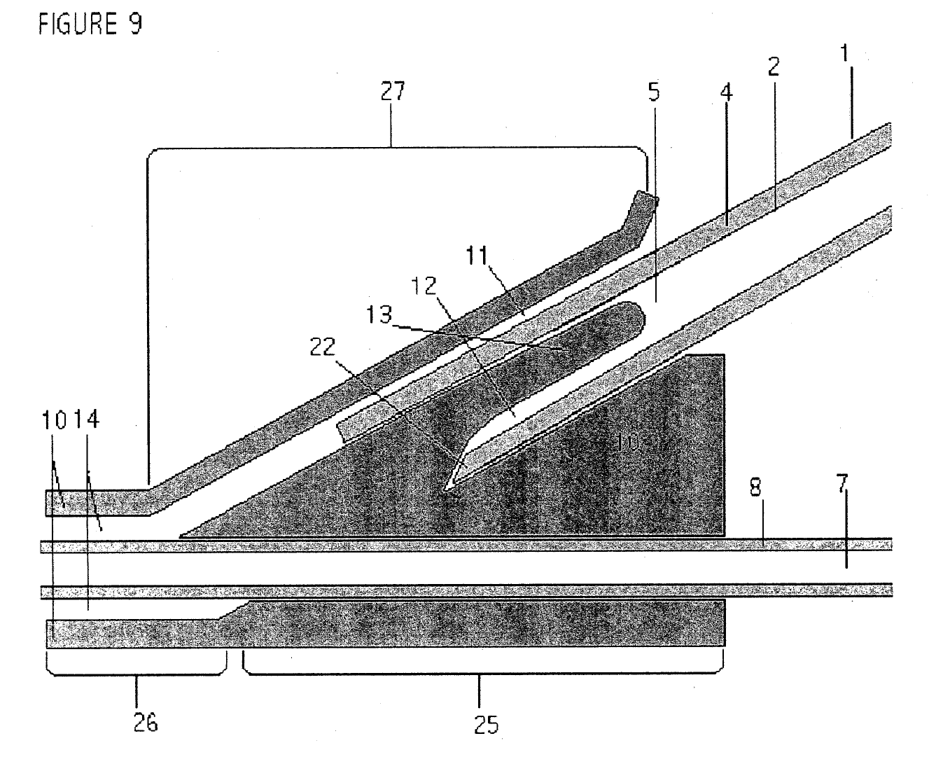

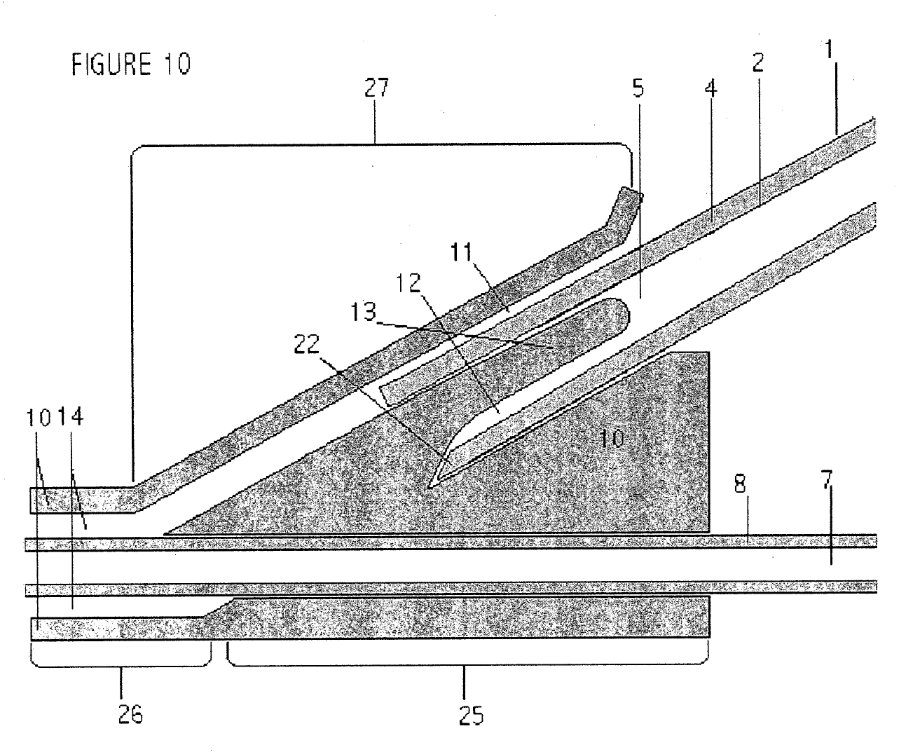

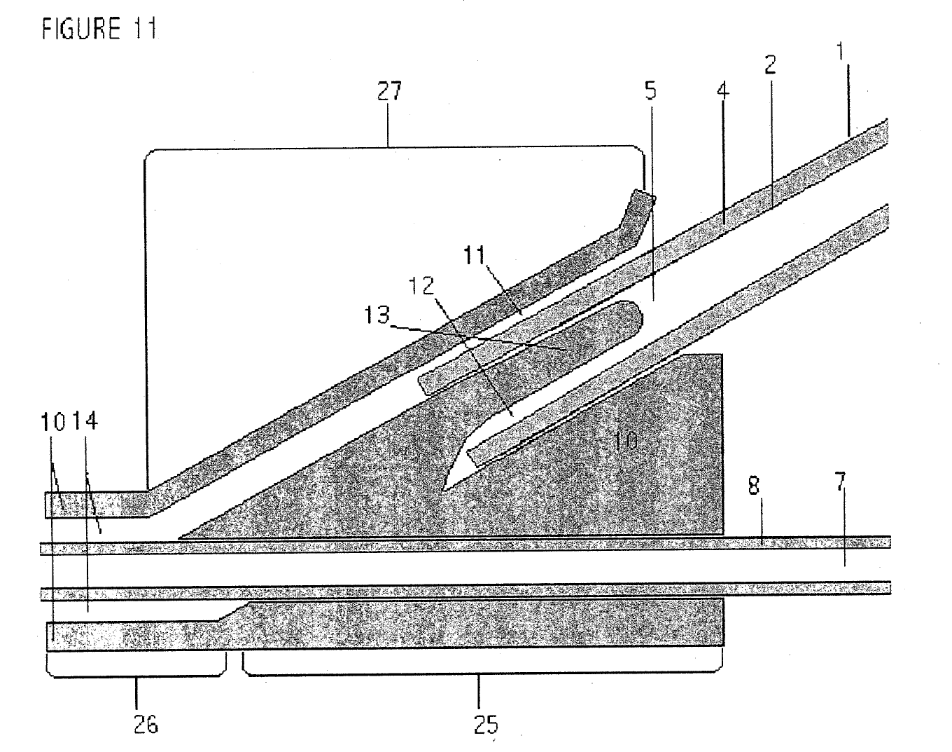

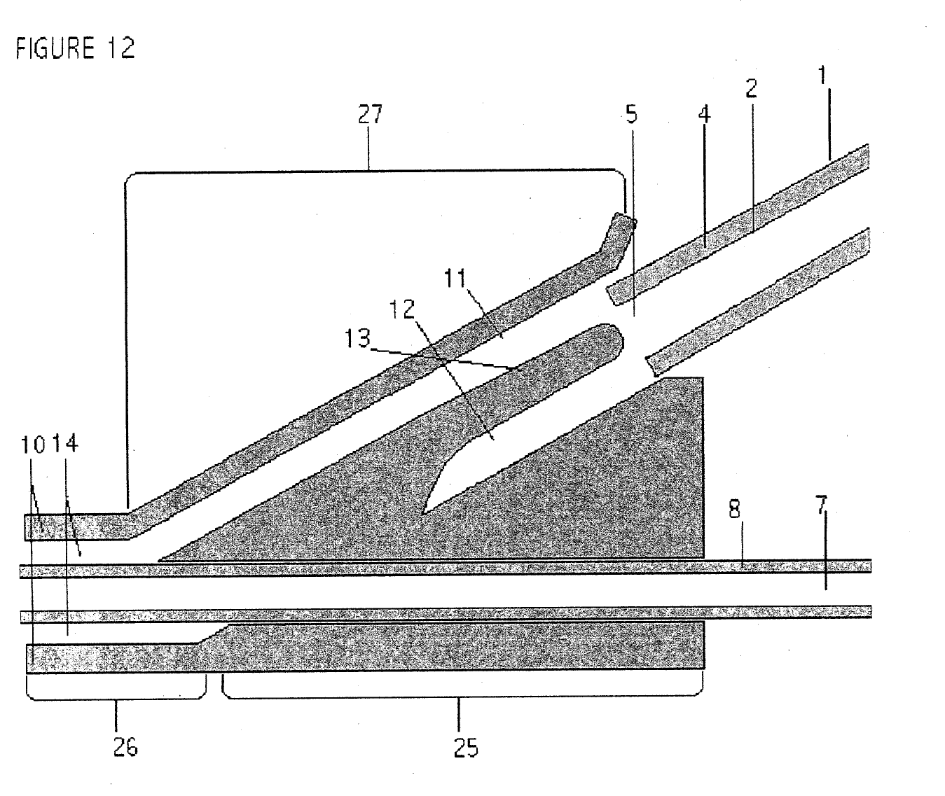

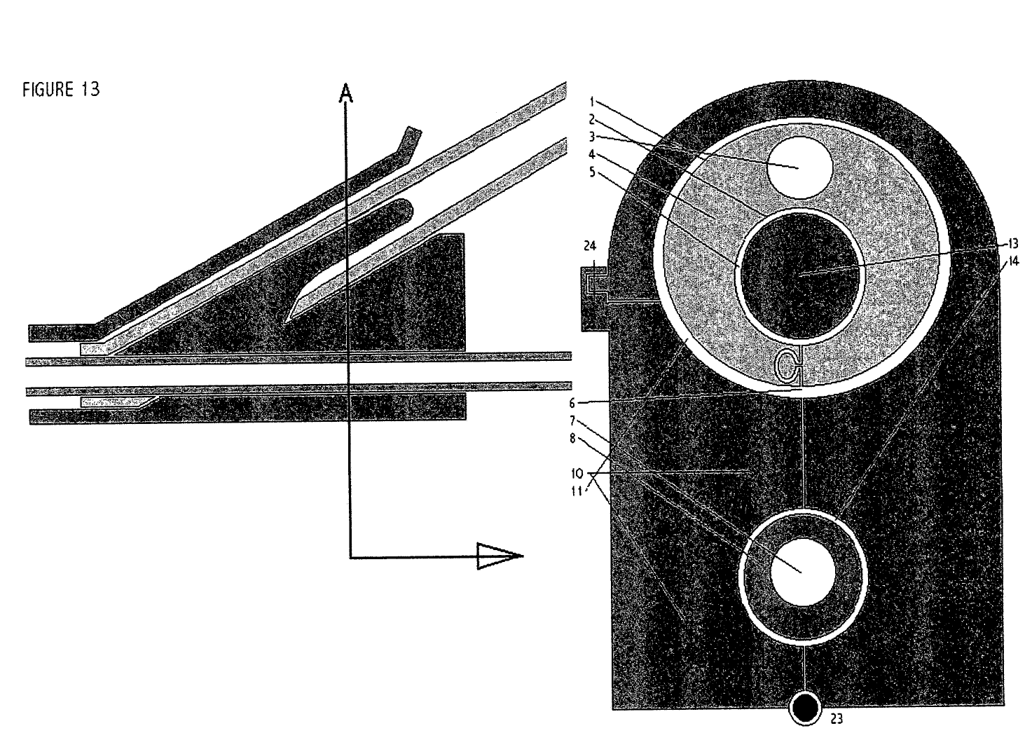

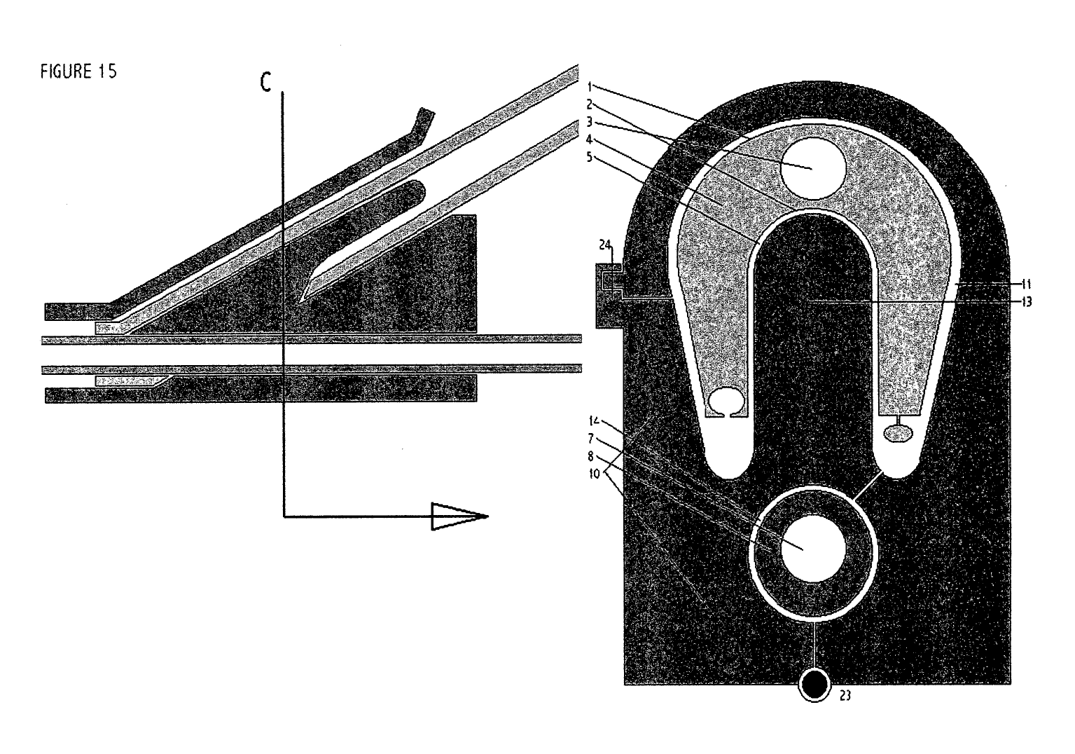

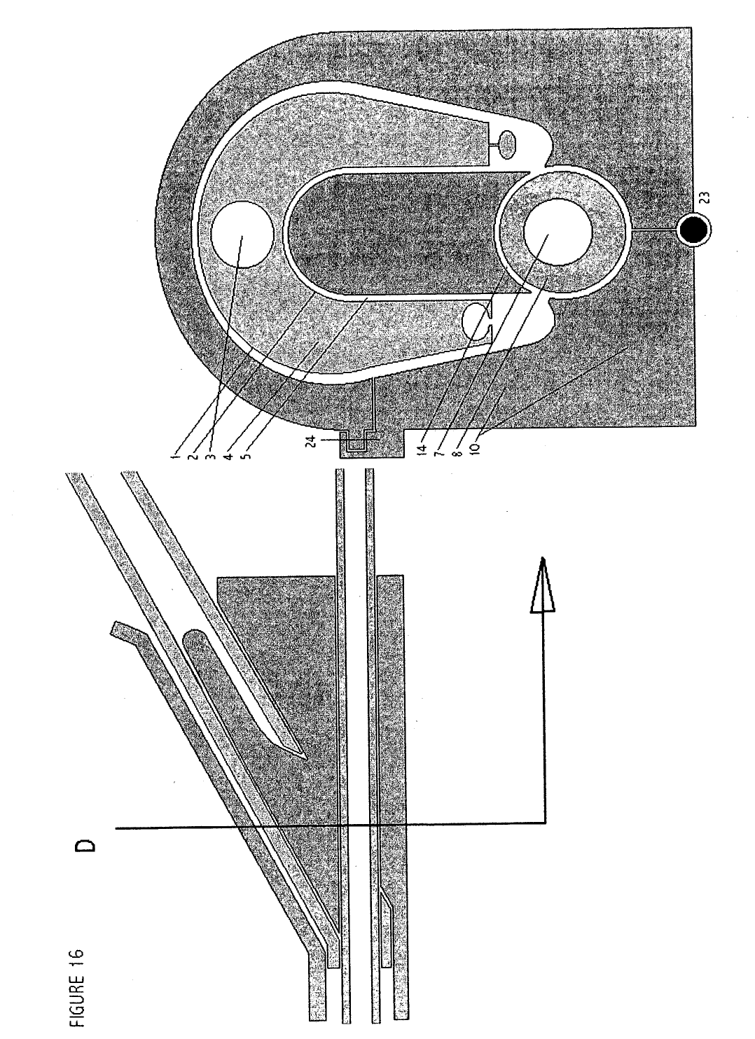

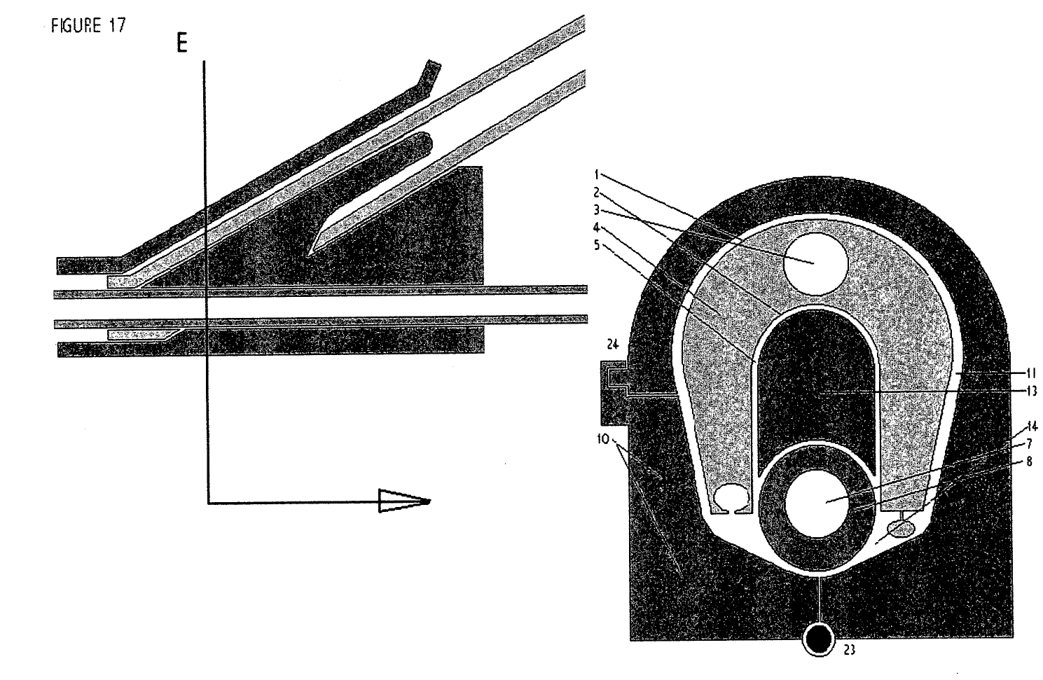

The invention in claim 1 (denoted 4 in figures) will have several things in common with catheters, tubes, dilators, shunts, drains, access devices or the like in current or future practice including its superficial appearance, material used for manufacturing, and the manufacturing process however it separates its self from these devices by having a sidewall defect (denoted 6 in FIGS. 2-6) that gives it the ability to transform from and to a closed and open tube state (FIGS. 7-19) to achieve several unique objectives not offered by catheters, tubes, dilators, shunts, drains, access devices or the like.

Currently catheters, tubes, dilators, shunts, drains, access devices or the like rely on the openings at the ends for exchange or positioning over guide wires a technique well know to the art. The current inventions will offer an alternative (FIGS. 7-19) where the invention of claim 1 (denoted 4 in figures) can be positioned over a catheter, tube, dilator, shunt, drain, access device or the like via a sidewall slit or defect (6 in FIGS. 2-6 and 13-21), without need for a traditional guiding device such as a wire resulting in bypassing at least two steps related to usage of the traditional guiding device. In addition to making procedures that currently rely on a wire less time consuming, less labor intensive it also will have the potential of making possible some procedures that are other wise difficult if not impossible to perform with conventional techniques that rely on guide wires.

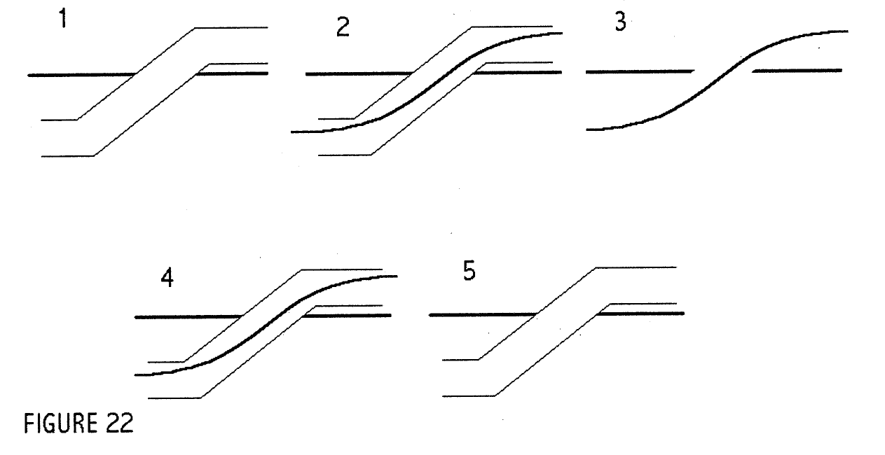

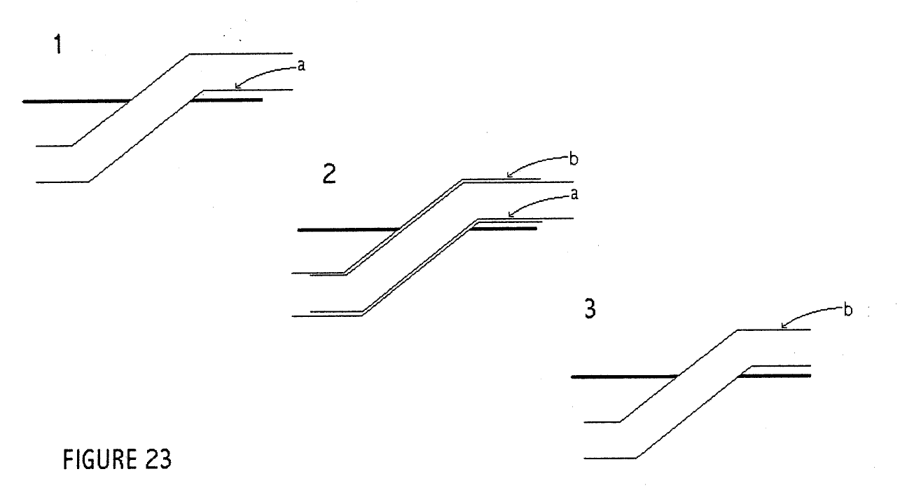

One area where the invention of claim 1 (denoted 4 in figures) is expected to have a positive impact is on exchange of catheters, tubes, dilators, shunts, drains, access devices or the like where the basic steps of conventional exchange require four basic steps that start with introducing a guiding device such as a wire, then removing the original catheter, tube, dilator, shunt, drain, access device or the like, then introducing a new catheter, tube, dilator, shunt, drain, access device or the like, then finally removal of the guiding device (FIG. 22). With the proposed inventions the two basic steps of exchange will be introducing the invention of claim 1 (denoted 4 in FIGS. 2-19) over the original catheter, tube, dilator, shunt, drain, access device or the like (denoted 8 in FIGS. 1, 3 and 7-19) followed by the second and final step of removing the original catheter leaving the invention of claim 1 which will function as a new catheter, tube, dilator, shunt, drain, access device or the like (FIG. 23). This will result in bypassing the first and last steps of the conventional method where the guiding device is introduced and removed.

Another area where the invention is expected to have a positive impact is having the ability to perform some procedures that are difficult or impossible with conventional methods that rely on traditional guiding devices such as a wire. The conventional methods rely on having a patent catheter, tube, dilator, shunt, drain, access device or the like that is able to accept a guiding device such as a wire, if these tubes are occluded introducing a wire is no longer easy and potentially impossible. For instance such a scenario is not infrequently encountered with tubes (gastric feeding tubes for example), catheters, shunts, drains, access devices or the like that becomes occluded and require exchange, which is frequently met with difficulty when trying to negotiate beyond the occlusion using a traditional access maintenance device such as a wire. With the invention of claim 1 (denoted 4 in FIGS. 2-19) it will be possible in some cases to introduce the invention of claim 1 (denoted 4 in FIGS. 2-19) over the occluded catheter, tube, shunt, drain, access device or the like (denoted 8 in FIGS. 1, 3 and 7-19) avoiding the need to pass a guide wire or the like across the occlusion as the invention of claim 1 will slide over the external aspect of the catheter, tube, shunt, drain, access device or the like (see FIGS. 7-19) not impeded by the occlusion inside the lumen. Additionally some catheters, tubes, shunt, drain or the like are designed with blind (closed) tips (some ventriculopertioneal shunt tubing for example), while exchange is made possible with invention of claim 1 (denoted 4 in FIGS. 2-19) in a minimally invasive fashion it is not possible with the conventional wire dependent method of exchange because the closed tip will prevent passage of the wire or the like but will not prevent deployment of invention of claim 1.

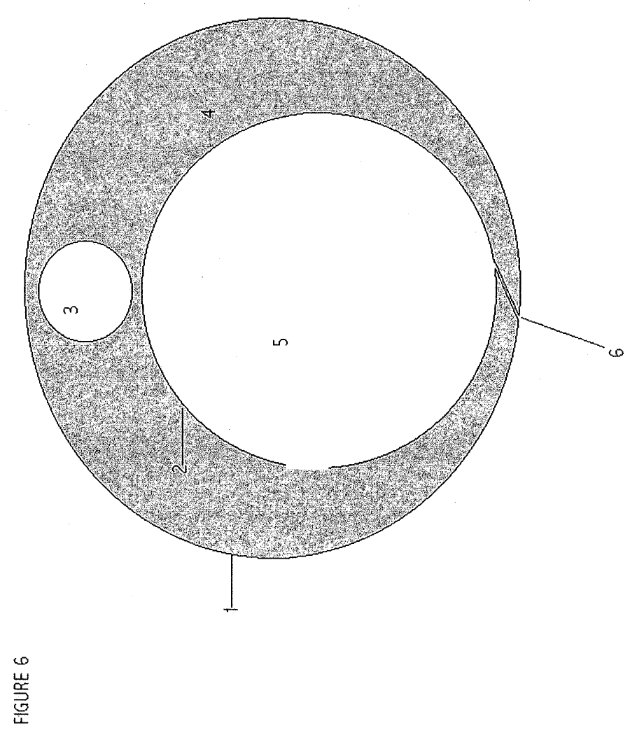

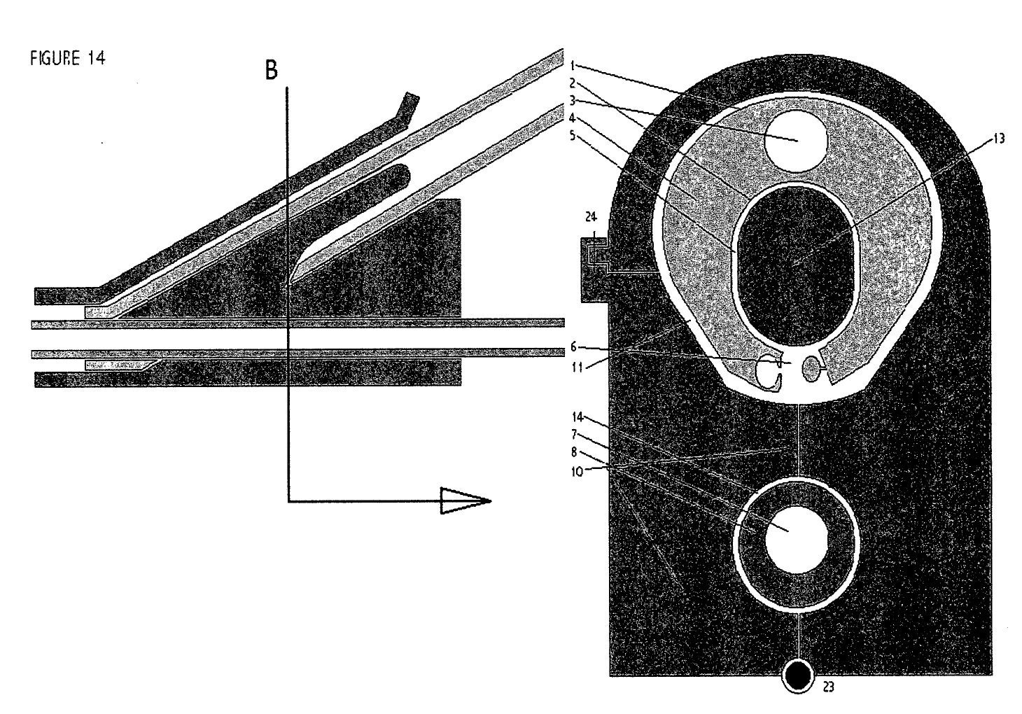

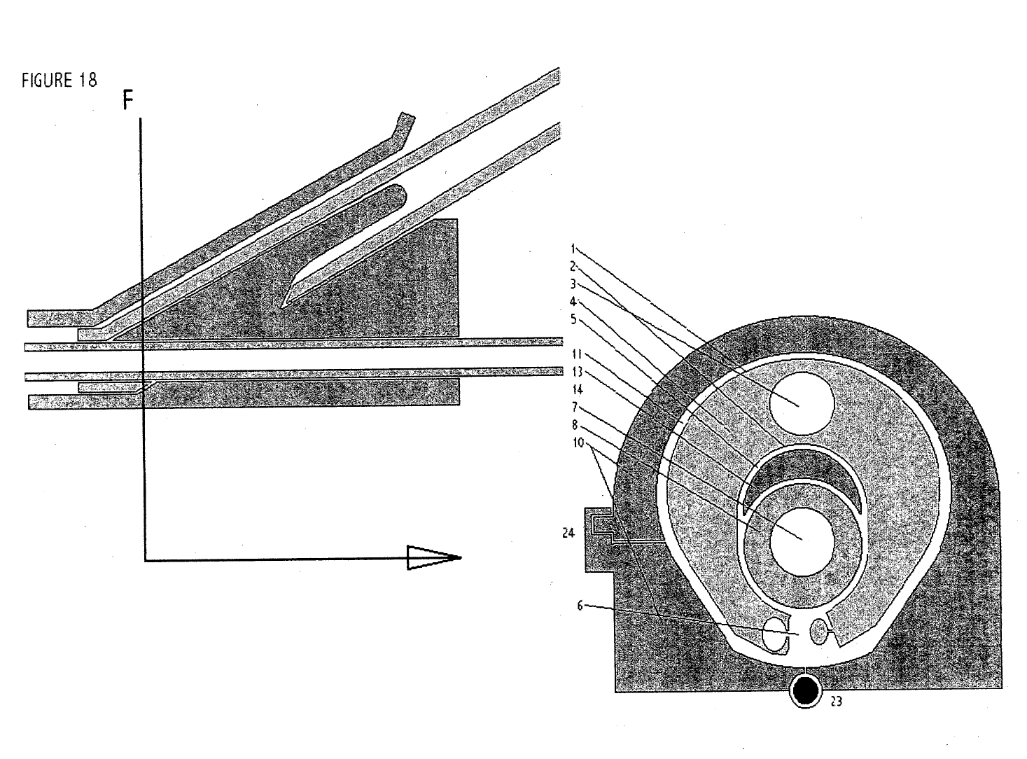

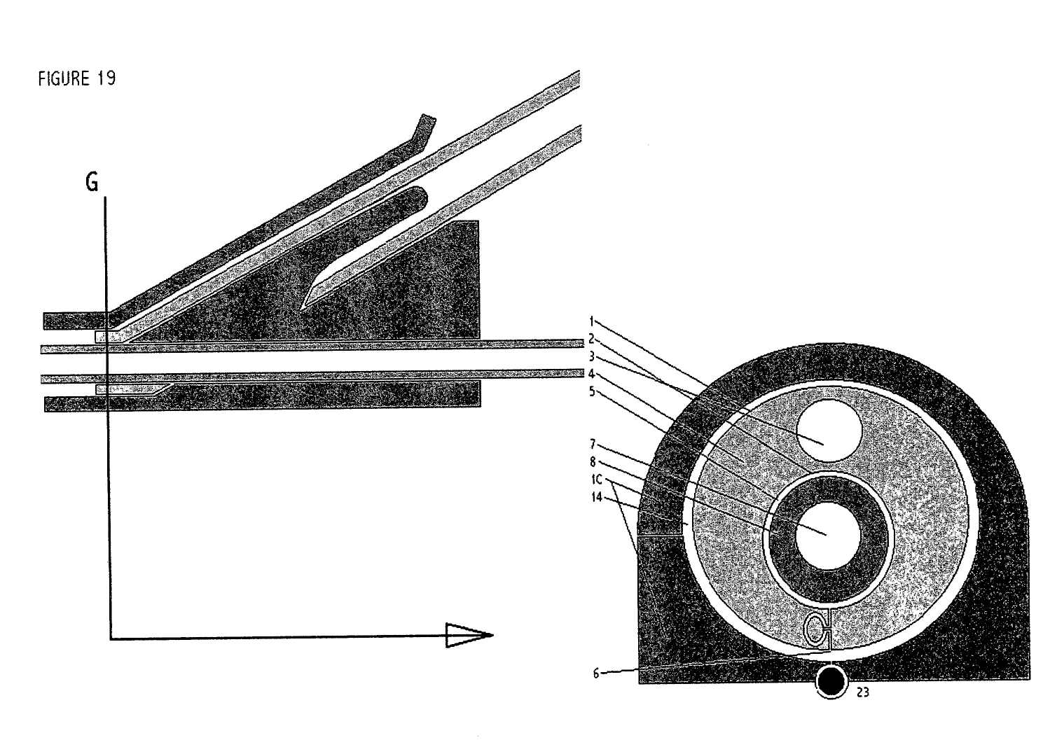

The invention of claim 1 (denoted 4 in FIGS. 2-19) features a lumen (5 in FIGS. 2-19) that will receive a guiding device (catheter, tube, dilator, shunt, wire, cable, drain, access device or the like) (8 in FIGS. 2-19). This lumen is different then the usual lumens present in all catheters, tubes, dilators, shunts, drains, access devices or the like known in the art in that it features a defect (6 in FIGS. 2-6 and 13-21) along all or part of its length where the internal surface of the lumen (2 in FIGS. 2-19) is in continuity with the external surface (1 in FIGS. 2-21) of the invention of claim 1 via the sidewall defect. This defect will allow a unique way of applying and removing (FIGS. 6-19) the invention of claim 1. Positioning the invention of claim 1 over a catheter, tube, dilator, shunt, drain, access device or the like (denoted 8 in FIGS. 2-19) relies on separating (FIGS. 14-17) the edges bordering the defect and then re-approximating (FIGS. 18, 19) them this will be achieved by the passage thru the invention of claim 2 (10 in FIGS. 7-19). The separation and re-approximation of the edges of the defect require that the invention of claim 1 be manufactured with flexibility to allow separation then re-approximation of the defect edges. Additionally it favorably will have a configuration memory where the edges (6 in FIGS. 2-6 and 13-21) assume an approximated (near closed) or closed neutral configuration upon removal of external forces used during the deployment of the invention of claim 1.

In addition to the lumen described above the device may feature optionally other elements that can be found in catheters, tubes, dilators, shunts, drains, access devices or the like such as having one or more lumens (3 in FIGS. 2-6 and 13-19), embedded reinforcement elements, inflatable devices, and/or ports for delivery of an ever increasing number of devices and materials all known to the art.

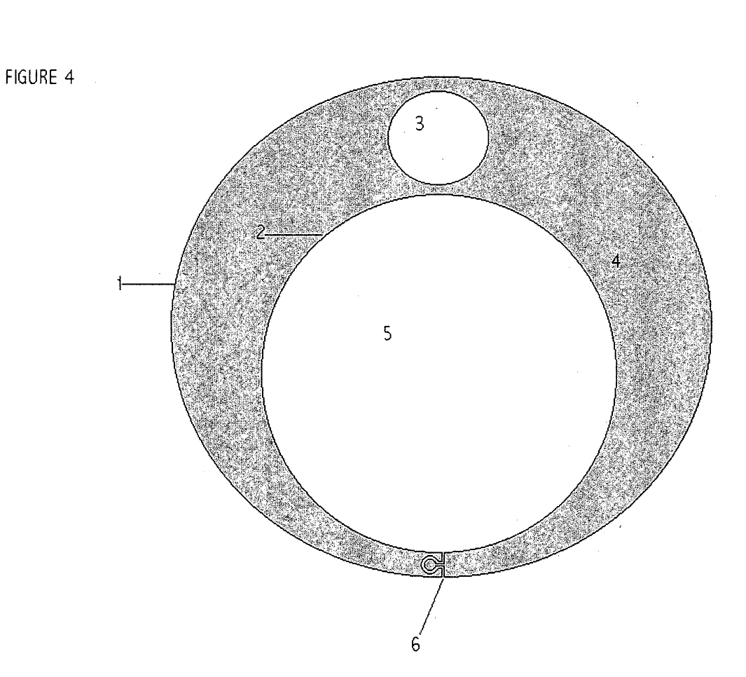

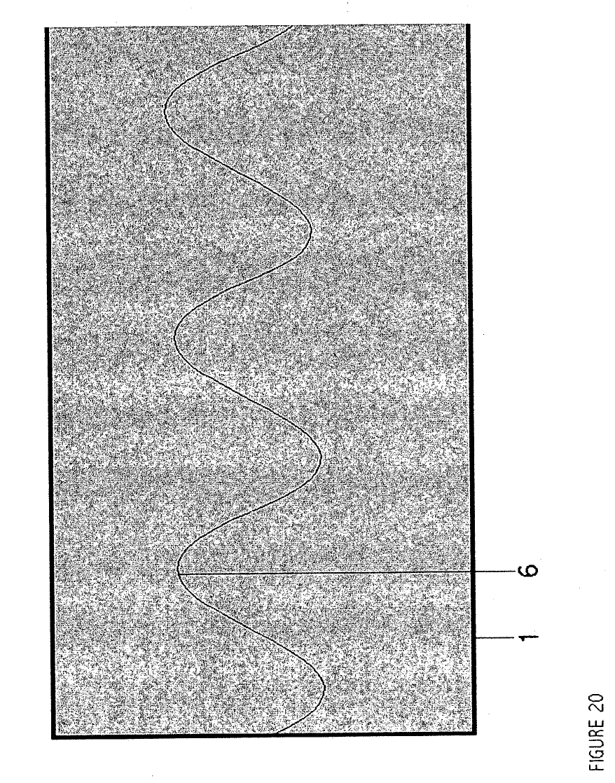

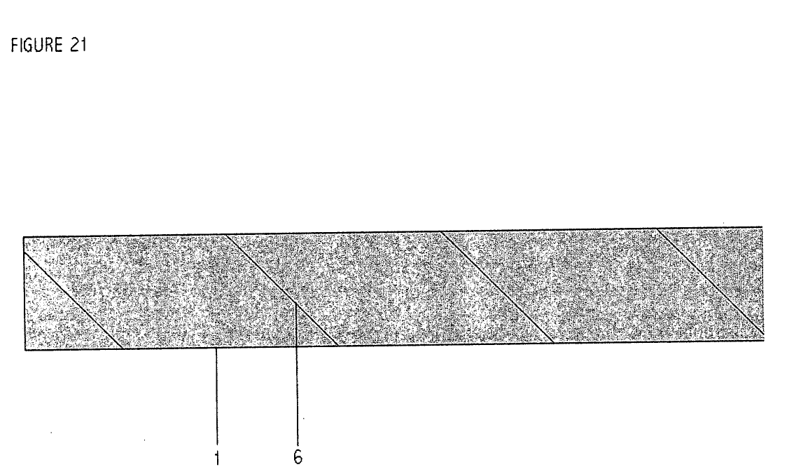

The sidewall defect (6 in figures) may be designed in several ways for instance they may be designed to have no overlap of the edges (FIG. 2)[claim 4], or may feature overlapping or interlocking edges [claim 3] some examples of which are demonstrated in FIGS. 4-6. The different designs are expected to offer variable degrees of ability in holding in their contents that may include catheters (denoted 8 in FIGS. 1, 3 and 7-19), dilators, tubes, drains, cables, access devices, shunt, wires, fluid, gas or the like. A different form of modification that is expected to offer added security in holding contents is an alteration that involves having a sidewall defect with a non-linear configuration [claim 5] (FIG. 20) such as a sinusoidal design, this design will necessitate that the sidewall defect (No# 6) be displaced a greater distance to allow free access to and from the lumen making it harder to loss containment of lumenal holdings. Another modification to the linear sidewall defect is to have a spiral sidewall defect that runs along all the circumference of the device(FIG. 21).

The invention of claim 1 (denoted 4 in FIGS. 2-19) can be manufactured using materials and manufacturing techniques and standards well known to the art in manufacturing existing catheters, tubes, dilators, shunts, drains, access devices or the like.

The invention of claim 2 (denoted 10 in figures) is a device that may be designed in several fashions one simplistic schematic representation is shown as 10 in FIGS. 7 thru 19. The function of this device is to create a force that separate then approximate or lock the free edges of the sidewall defect as the invention of claim 1 (denoted 4 in FIGS. 2-19) is advanced thru its special receiving port (denoted 11, 12 and 13 in FIGS. 7-19) in invention of claim 2 as illustrated by having a complex cavity that is designed to accept the invention of claim 1 (denoted 4 in FIGS. 2-19) with variable cross sectional configurations that exert forces on the device in claim 1 (denoted 4 in FIGS. 2-19) to causes the desired effect of opening and closing the sidewall defect (6 in FIGS. 2-6 and 13-21) as the devices of claim 1 is advanced. The components of the invention of claim 2 include:

A receiving port that accepts the invention of claim 1 (denoted 11, 12, 13 and 27 of the invention of claim 2) or modifications of claims 3 thru 5.

A cavity that accepts the desired guiding device such as a catheter, tube, dilator, shunt tube, wire, cable, drain, and access device or the like(denoted 25 in figures).

A portion of the device will have a cavity that will hold both the guiding device and the invention of claim 1 (or its modifications of claims 3 thru 5) when the two are fully engaged or deployed. This cavity will be continuous with both the receiving port and the cavity that accepts the desired guiding device. (Denoted 14 and 26 of the invention of claim 2)

It will also have movable components that will act like a door to allow access to and from the internal cavities. The device of claim 2 will have an open and locked states, the device also has locking components. One potential design is denoted 23 and 24.

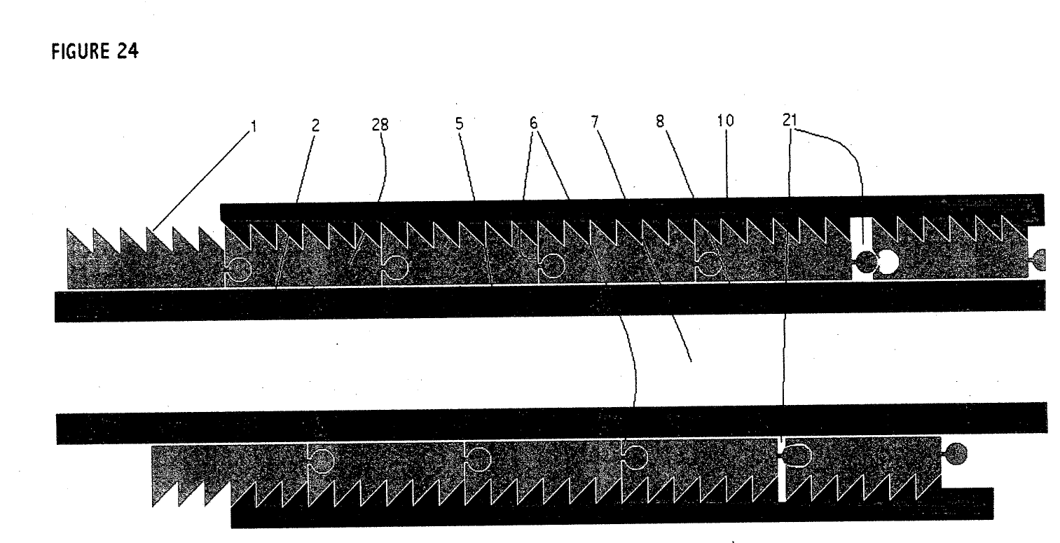

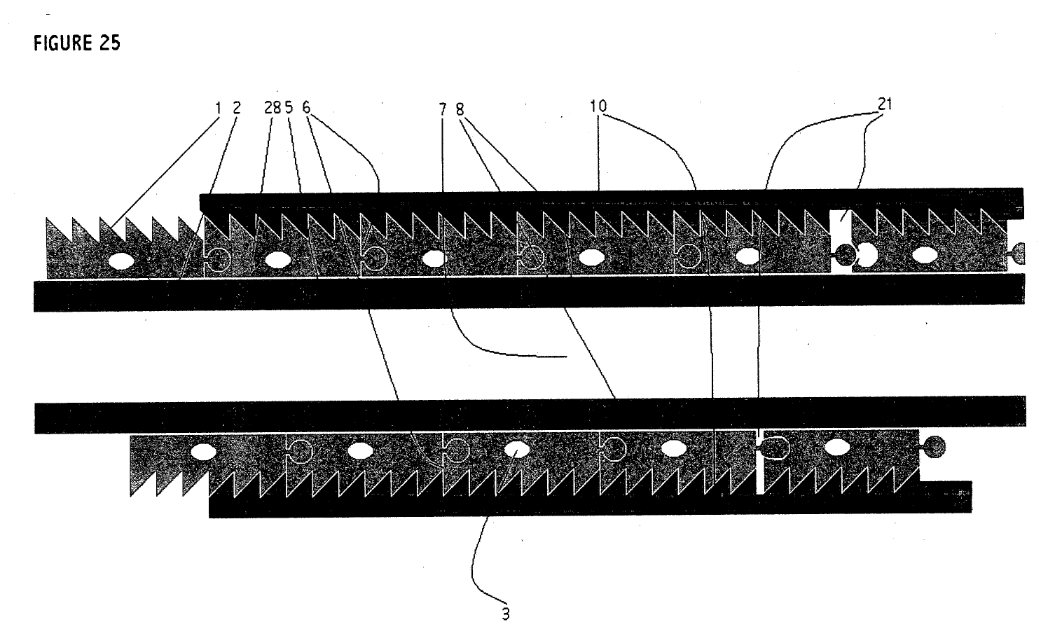

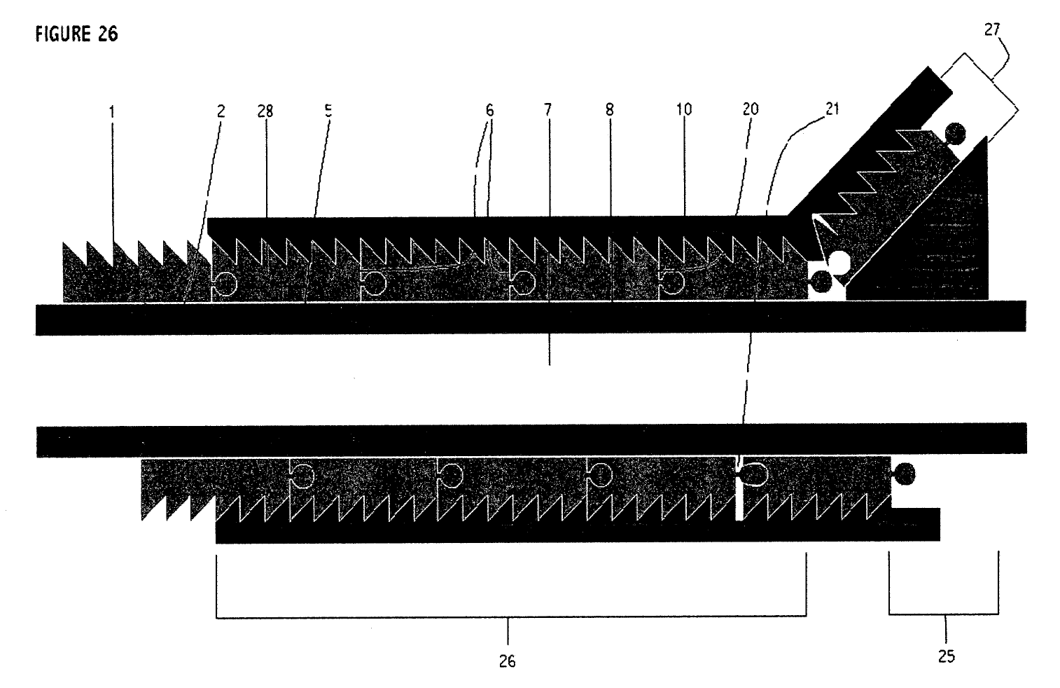

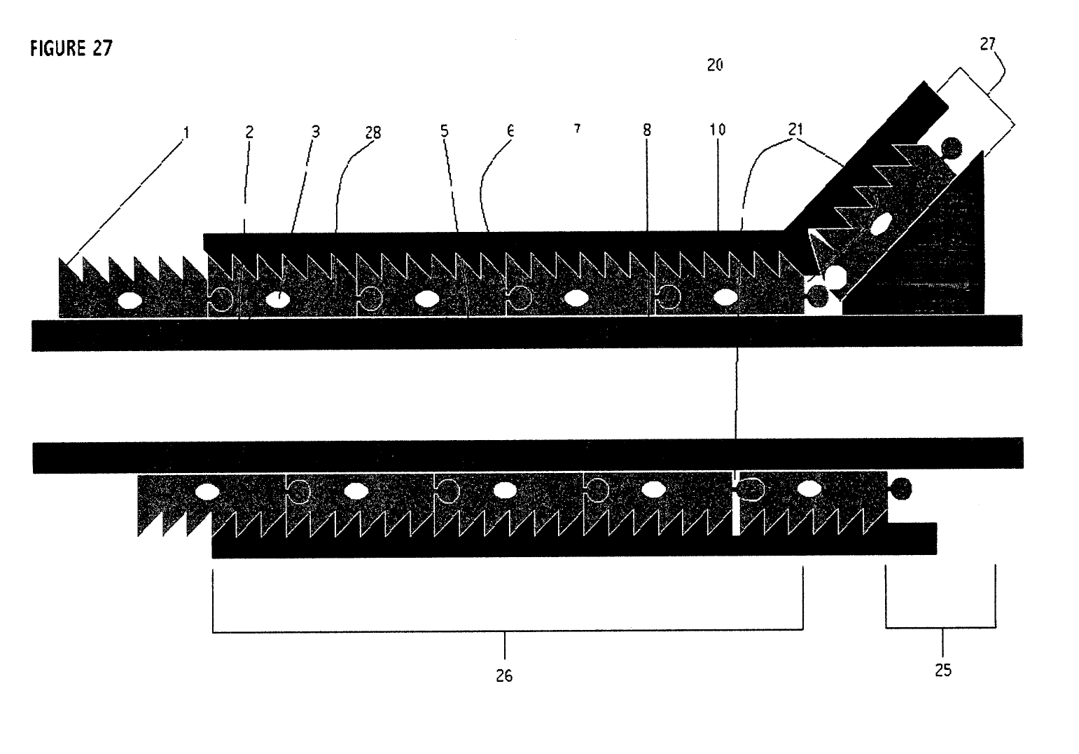

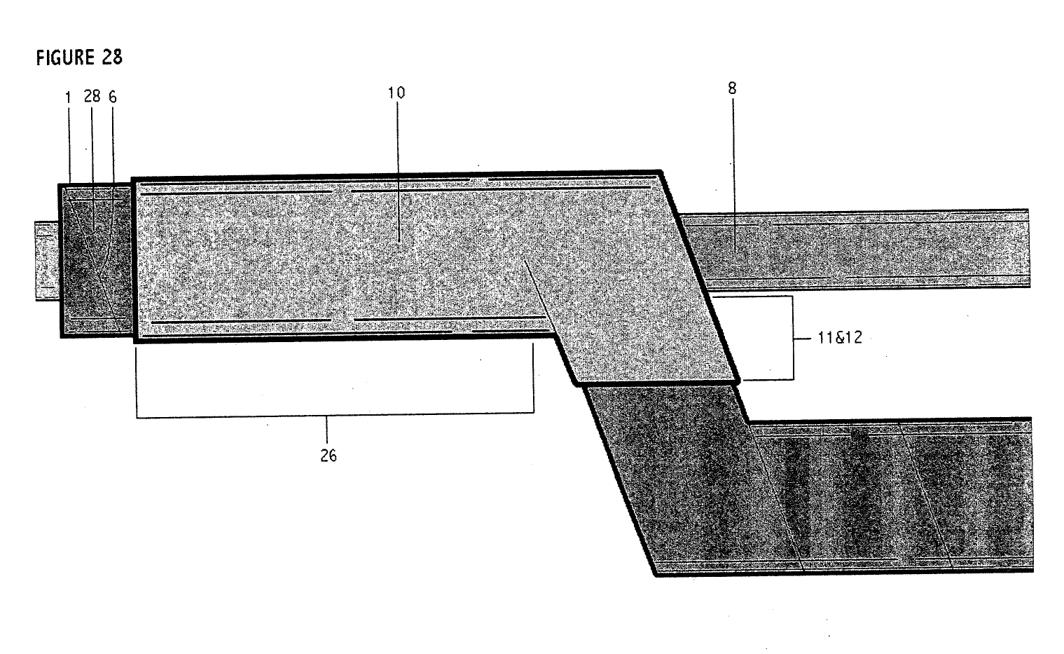

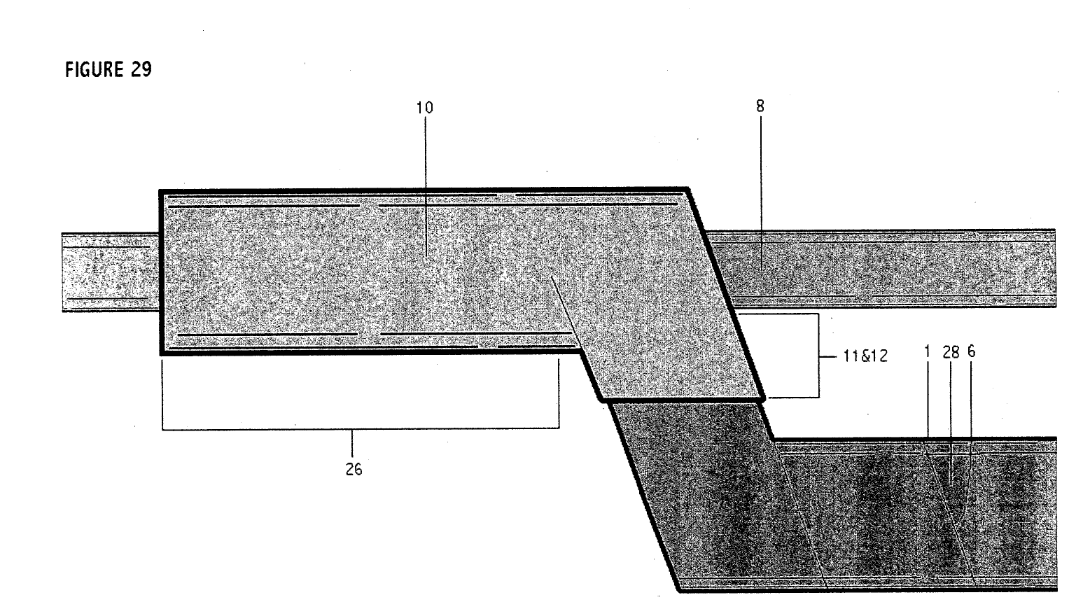

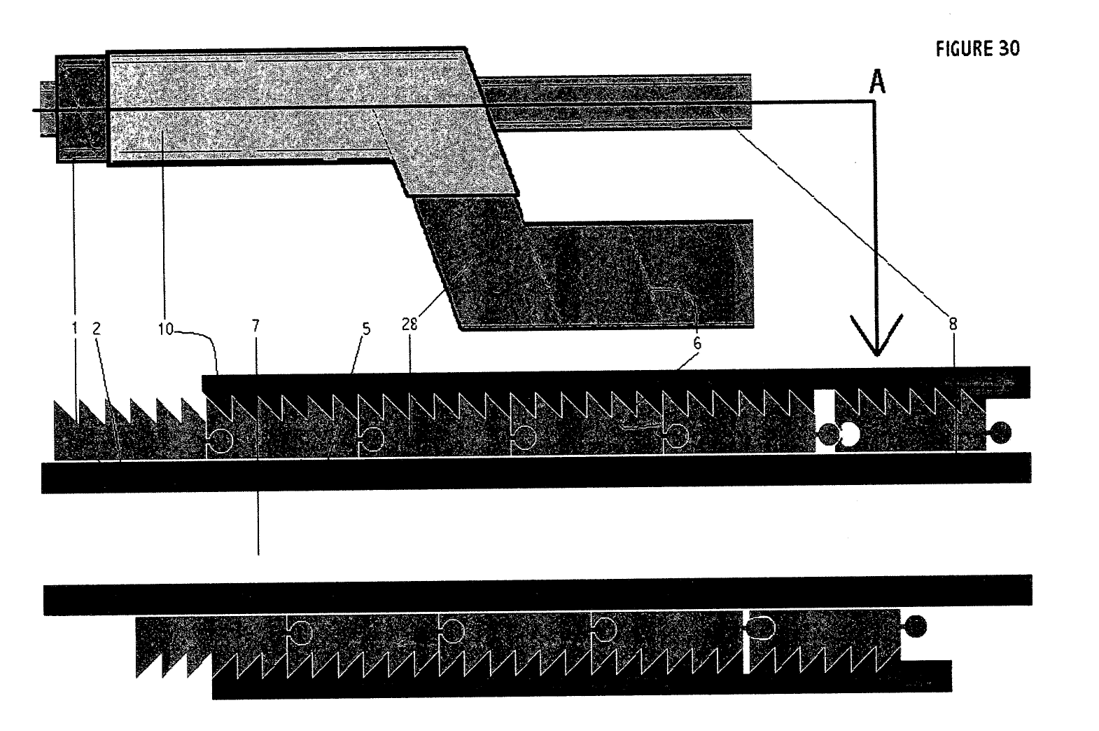

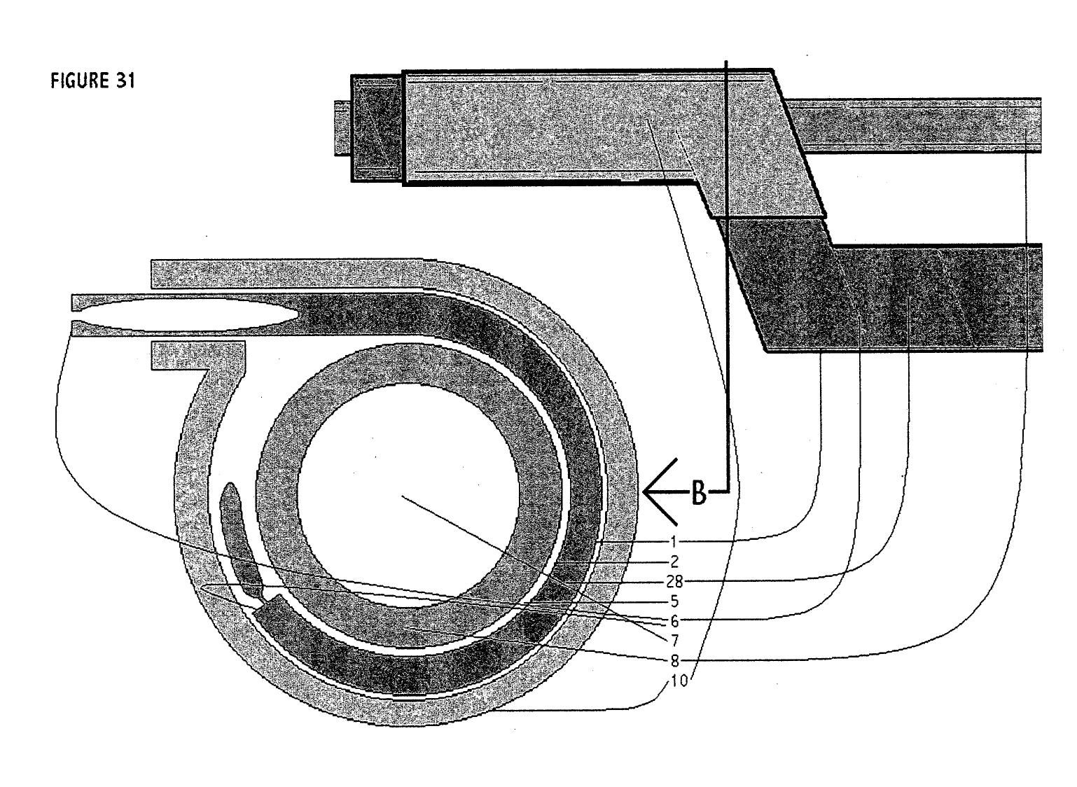

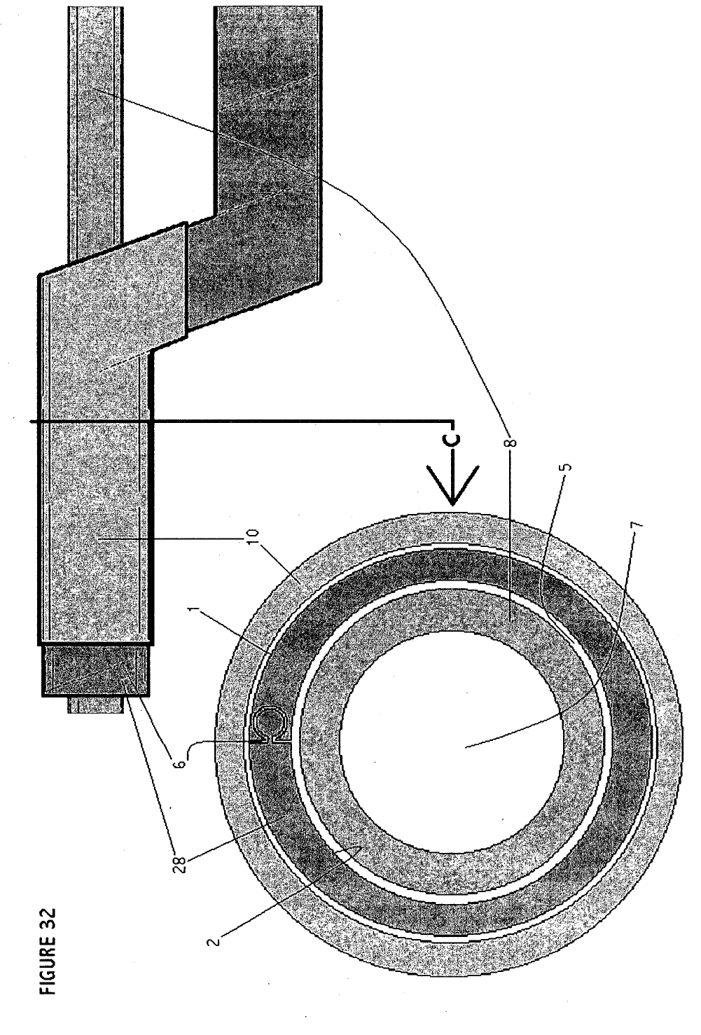

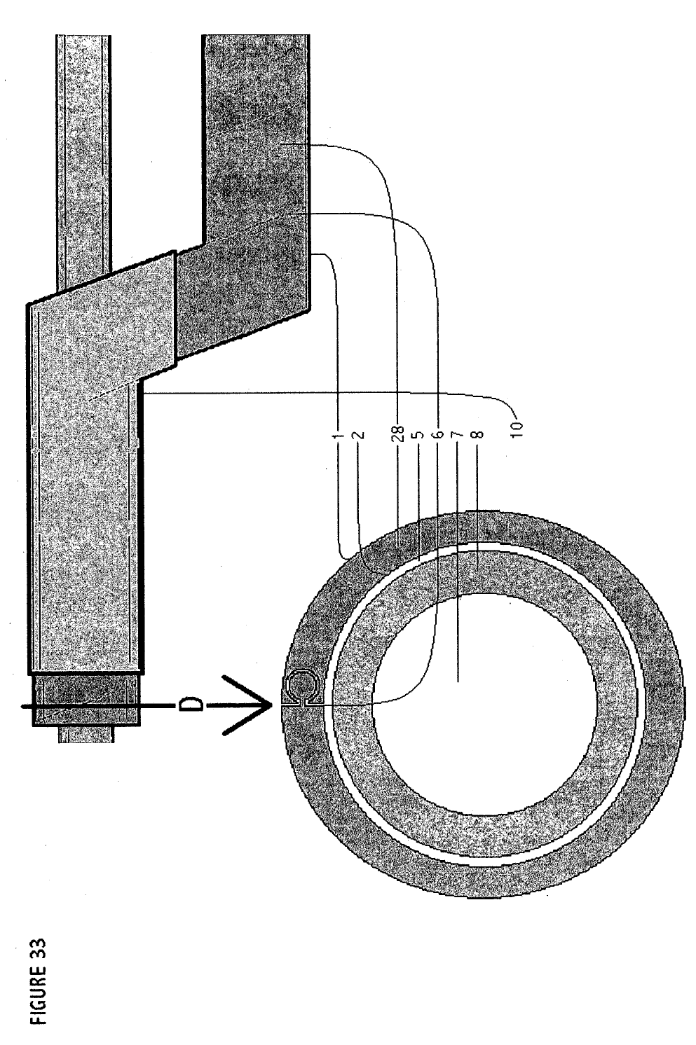

After placing and locking in the traditional catheter, tube, wire, cable, dilator, shunt, drain, access device or the like in its port (14 in FIGS. 7-19) in device of claim 2 (denoted 10 in FIGS. 7-19), a representation of how the invention of claim 1 (denoted 4 in figures) is deployed is illustrated in FIGS. 7 thru 19. The invention of claim 1 (denoted 4) is introduced into a special receiving port (denoted 11, 12 and 13 of the invention of claim 2), it is then advanced thru the invention of claim 2 which assists in deployment of the invention of claim 1 by receiving the device then separating the edges of the sidewall defect (denoted 6 of the invention of claim 1), this allows the invention of claim 1 to accept the traditional catheter, tube, drain, dilator, shunt, access device, wire or the like (denoted 8 in figures) in its lumen(denoted 5 in figures), then the invention of claim 2 will force the free edges of the invention of claim 1 to approximate and close its lumen after it has received the traditional catheter, tube, dilator, shunt, drain, access device, wire or the like in its lumen (denoted 5 in figures). The several potential modifications of the invention of claim 1 in some instances will require several designs of the invention of claim 2. For instance in the special case of having a spiral side wall defect (denoted 28 in FIGS. 24 thru 33) the design of the device of claim 2 illustrated in FIGS. 7 thru 19 will not be suitable for such a spiral side wall defect. One potential design of invention of claim 2 that will be suitable for a spiral side wall defect is illustrated in FIGS. 24 thru 33 and is denoted 10. In this illustrated design there is a spiral serrated internal surface of the device of claim 2 that complements the similarly designed external surface of the invention of claim 1 (denoted 1 on FIGS. 24 thru 33). The non-smooth surface in this special case will allow the device of claim 2 to exert a force along the longitudinal axis of device of claim 5 that permites approximation then locking of the free edges of the side wall defect.

While a preferred embodiment of the present inventions has been described, it should be understood that various changes, adaptations and modifications might be made therein without departing from the spirit of the inventions and the scope of the appended claims.

What is claimed is:

1] A catheter, tube, dilator, access device, shunt, drain like device that features at least one lumen, where the lumen surface communicates with the external surface of the device via a permanent or temporary sidewall defect along part or all of its length.

2] Device designed to separate and/or approximate (or rejoin) the free edges of the sidewall defect of device in claim 1.

3] Device described in claim 1 where the sidewall defect is designed to seal the lumen by overlapping and/or interlocking edges.

4] Device in claim 1 where the free edges of the sidewall defect merely approximate each other without creating a secure seal.

5] Device described in claim 1 where the sidewall defect is linear and/or nonlinear in design along the longitudinal axis and/or in cross section.
